(19) United States
(12) Patent Application Publication
(10) Pub. No.: US 2017/0128906 A1
(43) Pub. Date: May 11, 2017

(54) METHOD AND SYSTEM FOR CREATING LARGE VOLUMES OF HIGHLY CONCENTRATED PLASMA ACTIVATED LIQUID USING COLD PLASMA

(71) Applicant: EP Technologies LLC, Akron, OH (US)

(72) Inventors: Jeffrey S. Louis, Akron, OH (US); Tsung-Chan Tsai, Worthington, OH (US)

(21) Appl. No.: 15/345,753

(22) Filed: Nov. 8, 2016

Related U.S. Application Data
(60) Provisional application No. 62/252,720, filed on Nov. 9, 2015.

(51) Int. Cl. B01J 19/08; B04C 3/00; C02F 9/00; B01D 19/00

(52) U.S. Cl. CPC B01J 19/088; B01D 19/0057; B04C 3/00; C02F 9/00; C02F 1/50

(57) ABSTRACT

Exemplary embodiments of systems for generating large volumes of plasma activated liquids are disclosed herein. An exemplary system for creating a large volume of plasma-activated liquid includes a gas pump that moves a gas and liquid entrained in the gas, one or more plasma generators for generating plasma to activate at least one of the gas and the liquid entrained in the gas, a supply of liquid to be activated, a liquid aerator for creating an aerated liquid to be entrained in the gas, an activation chamber for activating the aerated liquid by contacting at least one of the aerated liquid or aerated liquid entrained in gas with plasma or plasma activated gas to form an activated liquid gas mixture. The exemplary system also includes a liquid gas separator positioned downstream of the activation chamber. The liquid gas separator separates at least a portion of the activated liquid gas mixture into an activated liquid and the gas. The activated liquid flows out of a first portion of the liquid gas separator and the gas flows out of a second portion of the liquid gas separator.

### RELATED APPLICATIONS

This application claims the benefits of, and priority to, U.S. non-provisional application Ser. No. 62/252,720 filed on Nov. 9, 2015, which is entitled Method and System to Create a Large Volume of Highly and is incorporated herein by reference in its entirety.

### TECHNICAL FIELD

The present invention relates generally to systems and devices for activating large volumes of liquid using cold plasma.

### BACKGROUND

It is known that plasma activated liquids have antimicrobial effects. However, many of the activated species have short half-lives and heretofore, no systems or methods have been developed that are capable of producing sufficient volumes of activated liquid to practically treat or decontaminate surfaces in an commercially feasible manner.

### SUMMARY

Exemplary embodiments of systems for generating large volumes of plasma activated liquids are disclosed herein. An exemplary system for creating a large volume of plasma-activated liquid includes a gas pump that moves a gas and liquid entrained in the gas, one or more plasma generators for generating plasma to activate at least one of the gas and the liquid entrained in the gas, a supply of liquid to be activated, a liquid aerator for creating an aerated liquid to be entrained in the gas, an activation chamber for activating the aerated liquid by contacting at least one of the aerated liquid or aerated liquid entrained in gas with plasma or plasma activated gas to form an activated liquid gas mixture. The exemplary system also includes a liquid gas separator positioned downstream of the activation chamber. The liquid gas separator separates at least a portion of the activated liquid gas mixture into an activated liquid and the gas. The activated liquid flows out of a first portion of the liquid gas separator and the gas flows out of a second portion of the liquid gas separator.

Another exemplary system for creating a large volume of plasma-activated liquid, includes a gas circulator that moves a gas, a plasma generator for generating plasma to activate the gas to form an activated gas, a supply of liquid to be activated, a liquid aerator for creating an aerated liquid, an activation chamber for activating the aerated liquid by contacting the aerated liquid with activated gas forming an activated liquid gas mixture, and a liquid gas separator positioned downstream of the activation chamber. The liquid gas separator separates at least a portion of the activated liquid gas mixture into an activated liquid and the gas and the activated liquid flows out of a first portion of the liquid gas separator and the gas flows out of a second portion of the liquid gas separator.

Another exemplary embodiment of system for creating a large volume of plasma-activated liquid includes a gas circulator that moves a gas through the system, a plasma generator for generating plasma to activate the gas to form an activated gas, a supply of liquid to be activated, a liquid aerator for creating an aerated liquid, an activation chamber for activating the aerated liquid by contacting the aerated liquid with activated gas forming an activated liquid gas mixture and a cyclonic separator positioned downstream of the activation chamber. The cyclonic separator separates at least a portion of the activated liquid gas mixture into an activated liquid and the gas.

### BRIEF DESCRIPTION OF THE DRAWINGS

These and other features and advantages of the present invention will become better understood with regard to the following description and accompanying drawings in which:

FIG. 1 illustrates a prior art device for generating small volumes of direct plasma activated water;

FIG. 2 illustrates a prior art device for generating small volumes of indirect plasma activated water;

FIG. 3 is an exemplary embodiment of a large volume plasma activated liquid generating system using a cyclonic separation device;

FIGS. 4 and 5 are an exemplary embodiment of a cyclonic separation device;

FIG. 6 is another exemplary embodiment of a large volume plasma activated liquid generating system using a cyclonic separation device;

FIG. 7 is another exemplary embodiment of a large volume plasma activated liquid generating system using a cyclonic separation device;

FIG. 8 is an exemplary embodiment of a large volume plasma activated liquid generating system using a venturi device;

FIG. 9 is another exemplary embodiment of a large volume plasma activated liquid generating system using a venturi device;

FIG. 10 is another exemplary embodiment of a large volume plasma activated liquid generating system using a venturi device;

FIG. 11 is another exemplary embodiment of a large volume plasma activated liquid generating system using a venturi device;

FIG. 12 is an exemplary embodiment of a large volume plasma activated liquid generating system using a bubbler device;

FIG. 13 is another exemplary embodiment of a large volume plasma activated liquid generating system using a bubbler device; and

FIG. 14 is another exemplary embodiment of a large volume plasma activated liquid generating system using a bubbler device.

### DETAILED DESCRIPTION

Plasmas, or ionized gases, have one or more free electrons that are not bound to an atom or molecule. Plasmas may be generated using a variety of gases including, air, nitrogen, noble gases (He, Ar, Xe, Kr, etc), oxygen, carbon dioxide and mixtures thereof under an electric field. In addition, non-thermal plasmas provide high concentrations of energetic and chemically active species. They can operate far from thermodynamic equilibrium with high concentrations of active species and yet remain at a temperature that is substantially the same as room temperature. The energy from the free electrons may be transferred to additional plasma components creating additional ionization, excitation and/or dissociation. Fluid that is contacted with plasma becomes “activated” and is referred to herein as plasma activated fluid, and in some embodiments, the plasma activated fluid is plasma activated water. If the fluid is in the liquid form, the liquid is plasma activated liquid. If the fluid is in a gaseous form, the fluid is a plasma activated gas. If the fluid is in a liquid gas mixture, the fluid is a plasma activated liquid gas mixture.

In some embodiments, plasmas may contain superoxide anions [O₂.⁻], which react with H⁺ in acidic media to form hydroperoxy radicals, HOO.:[O₂.⁻]+[H⁺]→[HOO.]. Other radical species may include OH. and NO. in gaseous or aqueous phase with the presence of air or gas. Properly treating water with non-thermal air plasma results in plasma activated water that may contain concentrations of one or more of atomic oxygen, ozone, H₂O₂, nitrates, nitrites, peroxynitrite, peroxynitrous acid, hydroxyl radicals and other active species. It is believed that the activated gas/droplet mixtures contains a significant amount of reactive species with short half-lives, such as for example, nitrogen species, such as nitrites and peroxynitrite.

Activating water with plasma to obtain plasma activated water is shown and described in U.S. Non-Provisional application Ser. No. 13/829,877 titled Sanitization Station Using Plasma Activated Fluid, filed on Mar. 14, 2013, which claims priority to U.S. Provisional Application Ser. No. 61/621,078 also titled Sanitization Station Using Plasma Activated Fluid, filed on Apr. 6, 2012 and U.S. Pat. No. 9,339,572 titled Methods of Making Solutions to Kill or Deactivate Spores Microorganisms, Bacteria and Fungus, filed on Mar. 15, 2013 and U.S. Non-Provisional application Ser. No. 13/842,574 titled Methods of Making Solutions to Kill or Deactivate Spores Microorganisms, Bacteria and Fungus, filed on Mar. 15, 2013 and U.S. Provisional Application Ser. No. 61/710,263 also titled Solutions and Methods of Making Solutions to Kill or Deactivate Spores, Microorganisms, Bacteria and Fungus, filed on Oct. 5, 2012, all of which are incorporated by reference herein in their entirety. Several other patents and applications disclose activating fluid, such as PCT Application Nos. WO 02/059046, titled Method of Activation of Chemically Pure and Potable Water and filed on Jan. 25, 2002; WO 2007/048806, titled Method for the Preparation of Biocidal Activated Water Solutions and filed Oct. 25, 2006; WO 2012/018891, which is titled Materials for Disinfection Produced by Non-Thermal Plasma and was filed on Aug. 3, 2011; and U.S. Pat. No. 7,291,314, titled Activated Water Apparatus and Methods and filed Dec. 20, 2001, and are incorporated herein by reference in their entirety. These applications disclose activating liquid with cold plasma, however, these systems do not readily lend themselves to the generation of large volumes of plasma activated liquid.

The exemplary embodiments shown and described herein utilize dielectric barrier discharge (“DBD”) plasma generators, however, the inventive concepts are not limited to DBD plasmas or DBD plasma generators. The applications incorporated herein disclose numerous plasma sources that may be used in accordance with the inventive concepts disclosed herein. Such plasma sources, may be, for example, corona discharge plasma, radio frequency plasmas, gliding arc plasmas, pulsed corona, direct current corona, and the like. Accordingly, plasma generators that generate these types of plasmas may be used in various embodiments disclosed herein. The methods disclosed herein may be used to activate many liquid formulations which are typically water based formulations.

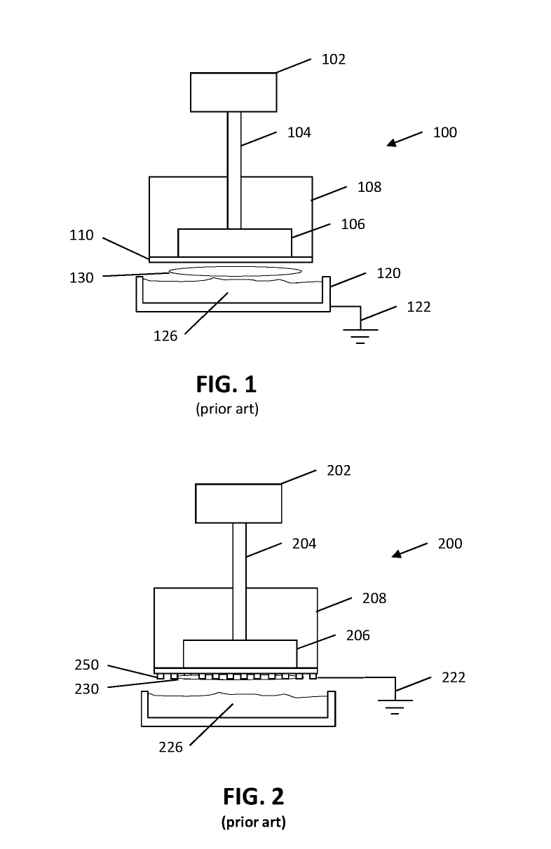

FIG. 1 illustrates a prior art method of activating water and other liquids using a dielectric barrier discharge (“DBD”) plasma generating system 100. The prior art plasma generating system 100 includes a high voltage source 102, a conductor 104, a housing 108, a high voltage electrode 106 and a dielectric barrier 110. The plasma generating system 100 also includes a container 120 which is grounded with grounding conductor 122. During operation, the high voltage source 102 is turned on and plasma 130 forms below the dielectric barrier 110. High voltage power source 102 may be a DC power source, a high frequency AC power source, an RF power source, a pulsed DC power source, a pulsed AC power source, a microwave power source or the like. The power supply can be pulsed with a duty cycle of 0-100% and pulse duration of 1 nanosecond up to 1 microsecond.

The plasma contacts the water or fluid 126 and activates the water or fluid 126. Fluid 126 activated by direct contact with plasma is referred to herein as “direct plasma activated fluid.” Because the plasma only contacts the surface of the fluid, this type of device does not readily lend itself to systems for activating the volume of fluid that would be necessary for commercial applications.

FIG. 2 illustrates an exemplary prior art system 200 for activating a fluid using indirect plasma. System 200 includes a high voltage power source 202. High voltage power source 202 may be a DC power source, a high frequency AC power source, an RF power source, a microwave power source, a pulsed DC power source, a pulsed AC power source or the like. The power supply can be pulsed with a duty cycle of 0-100% and pulse duration of 1 nanosecond up to 1 microsecond.

The exemplary system 200 includes a DBD plasma generator 208 connected to high voltage power source 202 by cable 204. Direct DBD plasma generator 208 includes a high voltage electrode 206 and a dielectric barrier 210 located between high voltage electrode 206 and the fluid 226 that is to be activated. A filter 250 is also included. Filter 250 is a conductive mesh that is grounded by grounding conductor 222.

During operation of system 200, when high voltage electrode 206 is energized, plasma 230 forms below the dielectric barrier 210, and the filter 250 (if the filter 250 is made of a conductive material and grounded) prevents charged ions and electrons from passing through and contacting the fluid 226 to be activated. Thus, only neutral species pass through and activate the fluid 226. This is typically referred to as “afterglow” or “indirect” plasma. In some embodiments, the fluid is water. Fluid 226 activated by afterglow that passes through, or is created through filter 250, is referred to “indirect plasma activated fluid.” Again, because the plasma only contacts the surface of the fluid, this type of device does not readily lend itself to systems for activating the volume of fluid that would be necessary for commercial applications.

In the exemplary embodiments disclosed herein the liquid being activated may be water. In some embodiments, the properties of the liquid may be altered prior to activation by plasma or indirect plasma to increase or decrease concentration of species, radicals and the like. For example, the pH of water may be adjusted to be acidic or basic. The pH may be adjusted by, for example, adding acid to the water prior to activation. The pH level may be lowered through the activation process. In one embodiment, the pH level of the activated water is about 2.0, in another the pH is between about 2.0 and 3.5, and in yet another is about 2.7. Still, in another the pH is less than about 3.0 and in another embodiment is less than about 2.0. In one embodiment, the pH is about 2.0.

In addition, the properties of the activated liquid may be adjusted during the activation process itself by altering the gas that is ionized at the electrode. For example, the gas that is ionized may be normal air, N₂, O₂, He, Ar, Xe, Kr, combinations thereof at various ratios, or the like. In some embodiments, one or more inert gases are used in the plasma generating process. In some embodiments, one or more noble gases are used in the plasma generating process, and in some embodiments, combinations of noble and other gases are used in the plasma generating process.

Further, additives may be added before or after the liquid is activated to increase efficacy or stabilization of the resulting solution. Other additives that may be used depending on the desired results include, for example, alcohol, silver salts, e.g., silver nitrate or silver chloride, or colloidal silver; zinc salts, e.g. zinc chloride, zinc lactate, or zinc oxide; suspensions containing metal nanoparticles; chlorhexidine; anionic, cationic, non-ionic and/or amphoteric surfactants; emulsifiers; hydrotropes; glycerol; chelating agents; alcohols; quaternary ammonium compounds, acids (organic or inorganic); bases; or surface tension decreasing agents.

The liquids may be a source of water, or of water with additional additives. In one embodiment, the liquid is tap water, however, the water may be distilled water, deionized water, tap water, filtered water, saline, water with acidic properties, water with basic properties or water mixed with additives such as, for example, alcohol. In addition, other additives may be used to optimize generation or increase performance and/or increase stability. These additives may include, for example chelators to reduce metal degradation; surfactants to improve penetration of the solution, to reduce the impact of organic load and/or buffers used to adjust the pH. In addition, in some embodiments corrosion inhibitors may be added, such as, for example, inorganic sulfates, inorganic phosphates. In some embodiments, a zeolite buffering system may be used. In some embodiments, one or more of these additives are added prior to activation of the water.

Methods and systems that use plasma to generate a large volume plasma-activated liquid (PAL) with high concentrations of activated species are disclosed herein. In some embodiments, the methods and systems create highly activated fluid in fog, vapor or small droplet form and separate the activated liquid to produce large volumes of highly activated liquid. In some embodiments, the methods and systems, disclosed herein, apply additional plasma after the plasma activated gas and vapor/or droplets have been mixed together to further enhance the activation of the liquid.

The liquid being activated can be a variety of different liquids. In some exemplary embodiments, the liquid can be water or water with additional additives. In some exemplary embodiments, the liquid can be an alcohol, such as ethyl alcohol, ethanol alcohol or isopropanol alcohol, diluted with water. Exemplary embodiments include formulations that contain water and ethanol mixtures. These formulations may contain up to about 70% ethanol, including up to about 60% ethanol, including up to about 50% ethanol, including up to about 40% ethanol, including up to about 30% ethanol, including up to about 20% ethanol, including up to about 10% ethanol.

In some exemplary embodiment, the liquid is tap water. The liquid may be distilled water, deionized water, tap water, filtered water, saline, water with acidic properties, and water with basic properties. In some exemplary embodiments, the additive is a stabilizer. Use of a stabilizer enables the activated liquid to retain its antimicrobial benefits for a longer period than would otherwise exist with formulations that do not have a stabilizer. An exemplary stabilizer is an alcohol, such as, for example, ethanol. In some exemplary embodiments, the properties of the liquid may be altered prior to activation by plasma or indirect plasma to increase or decrease concentration of species, radicals and the like.

The liquid can be mixed with additives to improve the antimicrobial efficacy against virus, bacteria and fungi. Non-limiting examples of additives that can be added to the liquid include alcohol (e.g., ethanol, isopropyl alcohol), hydrogen peroxide, nitrite (e.g. sodium nitrite), bio active oil (e.g., limonene, coconut oil, grape seed oil, olive oil, thyme oil), acid (e.g., acetic acid, citric acid, nitrous acid, hydrochloric acid), enzyme (e.g., superoxide dismutase, nitrate reductase); quaternary ammonium group (e.g., benzalkonium chloride, didecyldimethylammonium chloride), preservatives (e.g., methylparaben, propylparaben, phenoxyethanol), glycol (e.g., caprylyl glycol, propylene glycol), nonvolatile glycol ether (e.g., ethylene glycol n-hexyl ether, ethylene glycol n-butyl ether), and any combinations thereof.

The non-thermal plasma can be formed from any type of direct or indirect non-thermal plasma generator, such as a plasma jet, dielectric barrier discharge (DBD), DBD plasma jet, gliding arc, corona discharge, non-thermal arc discharge, pulsed spark discharge, hollow cathode discharge, glow discharge, and the like. The voltage waveform generated by the plasma power supply can be DC, pulsed DC, pulsed AC, AC sinusoidal, RF, microwave and the like. The plasma can be driven by ambient air. The plasma can also be driven by feeding gas. Non-limiting examples of feeding gas that may be used include noble gasses (eg. helium, argon), molecular gasses (e.g. oxygen, nitrogen), gas carrying evaporated liquids, or combination thereof.

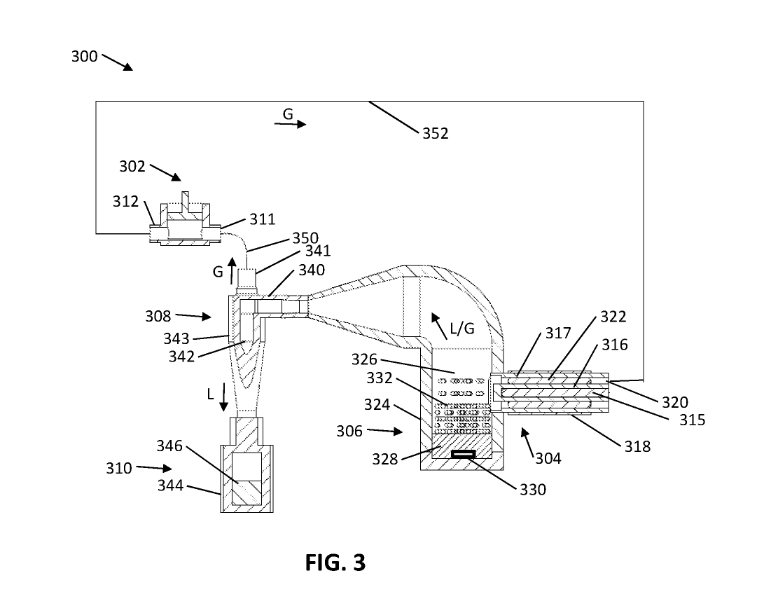

FIG. 3 is an exemplary embodiment of a large volume plasma activated liquid generating system 300 using cyclonic separation device. The exemplary system includes a vacuum pump 302, a plasma generator 304, and activation chamber 306, a cyclonic separator 308 and an activated liquid collection chamber 310. Vacuum pump 302 may be any type of vacuum pump 302 capable of generating the required gas flow through device 300 at the desired speed and pressure. The inlet 311 of vacuum pump 302 is connected to gas outlet 341 of cyclonic separator 308 by conduit 350. The outlet of vacuum pump 302 is connected to the gas inlet of plasma generator 304 via conduit 352. The gas flows in direction “G”.

Plasma generator 304 is a DBD plasma generator and includes a high voltage electrode 315 that is at least partially surrounded by a dielectric barrier 316. Plasma generator 304 includes a second dielectric barrier 317 surrounded by second electrode 318, which is a ground electrode. A gas inlet passage 320 allows gas to flow through plasma generator 304 into plasma activation chamber 306. High voltage electrode 315 is connected to a high voltage power source (not shown) which is used to generate plasma 322 within the gas flow chamber 302.

Activation chamber 306 generates an aerated liquid 332. The term aerated liquid includes liquid mists, fog, small droplets, vapor and the like. The aerated liquid 332 is contacted by the plasma activated gas flowing out of plasma generator 304. The aerated liquid 332 is activated by the plasma activated gas. In this exemplary embodiment, activation chamber 306 generates a mist of low mass small droplets (or vapor) utilizing an aerator 330, such as, for example, one or more piezoelectric disc, which are located in, on, or near liquid 328 which is being activated. Liquid 328 may be any type of liquid, such as, for example, those described above.

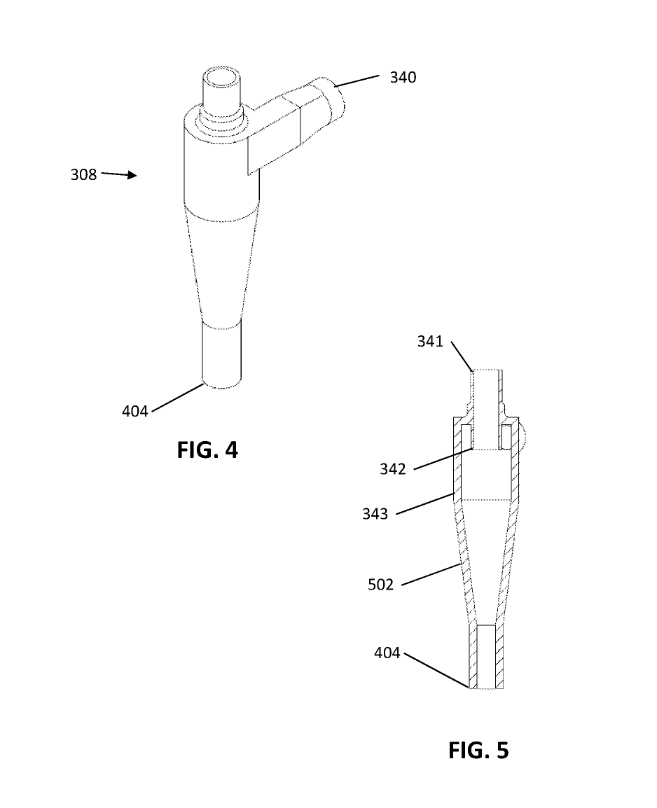

The activated liquid/gas mixture flows in direction L/G and flows into the inlet 340 of cyclonic separator 308 (see also, FIGS. 4 and 5). The inlet of the cyclonic separator is tangential to the cylindrical top portion 343. The activated liquid/gas mixture is separated through cyclonic separation. Cyclonic separation is a method of removing liquid from gas through vortex separation. The rotational effects, centrifugal forces and gravity to separate the fine droplets of liquid from a gaseous stream. In this exemplary embodiment, a high speed rotating liquid/gas flow is established within a cylindrical or conical container (i.e. a cyclone). The flow is typically in a helical pattern, beginning at the top 343 (wide end) of the cyclone and ending at the bottom (narrow) end 502 before the gas exits the cyclone in a straight stream up through the center of the cyclone and out the top in direction G. The activated liquid 346, which is denser than the gas in the rotating stream, has too much inertia to follow the tight curve of the gas stream, and strikes the outside wall. The activated liquid 346 then falls to the bottom of the cyclonic separator 308, out of the outlet 404 and into activated liquid collection container 310.

In a conical system, as the rotating flow moves towards the narrow end 502 of the cyclone, the rotational radius of the stream is reduced, thus separating smaller and smaller droplets. This exemplary cyclonic separation device 300 recycles the gas. Accordingly, any ozone generated by the plasma generator 304 is contained within the system. In addition, it is not necessary for the cyclonic separator 308 to remove all of the liquid entrained in the gas prior to recirculating the gas through the system. Liquid entrained in the gas that flows through the plasma generator is further activated or reactivated by the plasma. In some embodiments, it is desirable for the recycled gas to contain liquid particles or droplets as it recirculates through the system. The activated liquid 346 may be removed from, or piped out of, the system while the system is running, or the system may be stopped to remove the activated liquid 346.

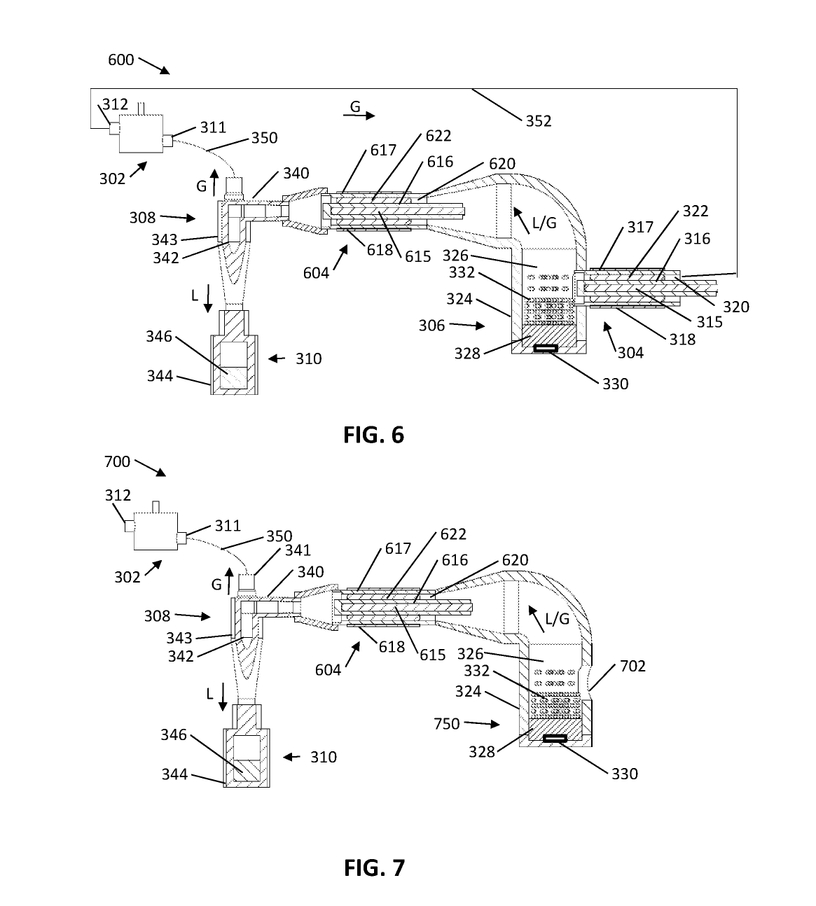

FIG. 6 illustrates another exemplary embodiment of a large volume plasma activated liquid generating system 600 using a cyclonic separation device 308. System 600 is similar to system 300 and like parts are not re-described herein. Large volume plasma activated liquid generating system 600 contains a second plasma generator 604. Plasma generator 604 is a DBD plasma generator and includes a high voltage electrode 615 that is at least partially surrounded by a dielectric barrier 616. Plasma generator 604 includes a second dielectric barrier 617 surrounded by second electrode 618, which is a ground electrode. High voltage electrode 615 is connected to a high voltage power source (not shown) which is used to generate plasma 622 within the gas flow chamber 602. A gas inlet passage 620 allows the activated liquid/gas (L/G) to flow through plasma generator 604 further activating the liquid/gas before if flows into cyclonic separator 308. The remainder of the process is the same as described above.

FIG. 7 is another exemplary embodiment of a large volume plasma activated liquid generating system using 700 a cyclonic separation device. System 700 is similar to system 600 and like components and functions are not re-described herein. Large volume plasma activated liquid generating system 700 does not include plasma generator 304 or conduit 352. In this exemplary embodiment, vacuum pump 302 causes air to flow into aerated liquid forming chamber 750, aerated liquid forming chamber is similar to activation chamber 306 and contains similar components. With the removal of plasma generator 304, air may flow through opening 702 to carry aerated liquid 332 into plasma generator 604. In some embodiments, a valve is included in opening 702 to regulate the volume of air that flows into chamber 750. In this exemplary embodiment, vacuum pump 302 discharges the air into the atmosphere through vacuum pump 302 outlet 312. In some embodiments, vacuum pump 302 is routed to opening 702 and the gas is recirculated.

In all of the embodiments, described above, the exhaust gas out of the vacuum pump 302 could also pass through an ozone destruction device (not shown) before it is recirculated back into the system or discharged into the atmosphere. In some embodiments, only part of the exhaust gas is routed back into the plasma generator 304. In addition, the system may include a valve to control the amount of exhaust gas being routed back into the plasma generator 304. In some embodiments, a feedback control loop to control the valve based on one or more parameters may be included. The system may also include one or more sensors to detect/measure one or more parameters and provide a signal to a valve controller indicative the value of the parameter. In addition, the above systems may use ambient air or one or more other gases, such as, for example, those listed above.

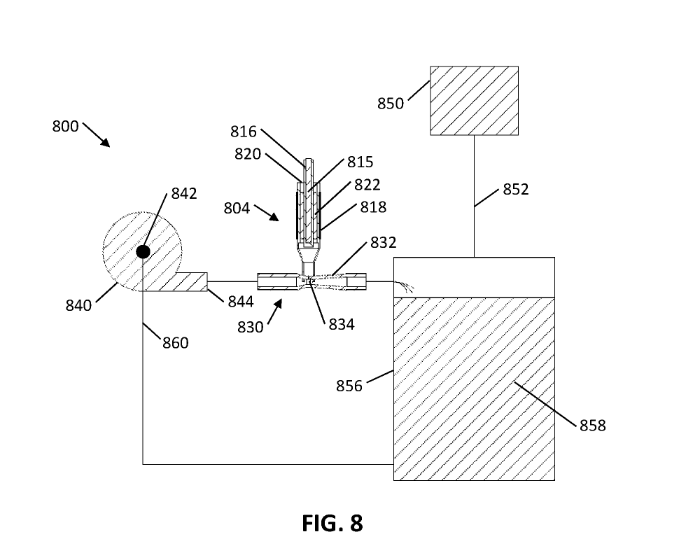

FIG. 8 is another exemplary embodiment of a large volume plasma activated liquid generating system 800 using a venturi device 830. System 800 includes a pump 840, plasma generator 804, venturi tube 830, tank 858 and ozone destruction device 850.

Plasma generator 804 is a DBD plasma generator and includes a high voltage electrode 815 that is at least partially surrounded by a dielectric barrier 816. Plasma generator 804 includes a second dielectric barrier 817 surrounded by second electrode 818, which is a ground electrode. A gas inlet passage 820 allows gas to flow through plasma generator 804 into a gas inlet 834 of venturi tube 830. High voltage electrode 815 is connected to a high voltage power source (not shown) which is used to generate plasma 822 within the gas flow chamber 802.

Pump 840 pump includes a pump inlet 842 and a pump outlet 844. Pump 840 pumps liquid through venturi tube 830, which has a reduced cross-section prior to gas inlet 834. Venturi tube 830 expands after the reduced cross-section, thereby generating suction at gas inlet 834. The suction draws in plasma activated gas to mix with the liquid 858 and activate the liquid 858. The activated liquid gas mixture flows into tank 856. Conduit 860 draws liquid 858 from tank 856 thereby recirculating activated liquid 858. The liquid may be piped out of tank 856, or tank 856 may be removed to use the activated liquid. In fluid communication with tank 856 is an ozone destruction device 850 that may be used to destroy ozone generated by plasma generator 804 before it is discharged to the atmosphere. In this exemplary embodiment, the gas flowing into plasma generator 804 is ambient air, however any gas, such as those identified above, may be used based on the desired characteristics of the activated liquid.

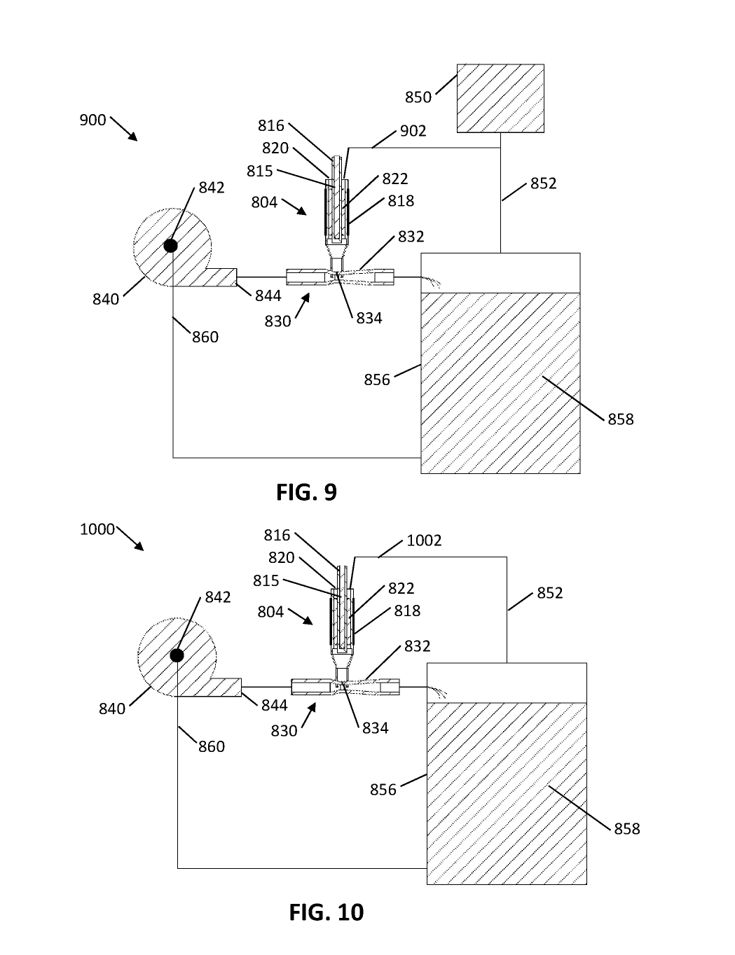

FIG. 9 is another exemplary embodiment of a large volume plasma activated liquid generating system 900 using a venturi device 830. System 900 is similar to system 800 except system 900 recycles gas in the system. Accordingly, the gas fed into plasma generator 804 may be ambient air or another gas, such as one or more of the gases disclosed above.

Similarly, FIG. 10 is another exemplary embodiment of a large volume plasma activated liquid generating system 1000 using a venturi device 830. System 1000 is similar to system 900 except system 1000 does not include an ozone destruction unit. The gas fed into plasma generator 804 may be air or another gas, such as one or more of the gases disclosed above.

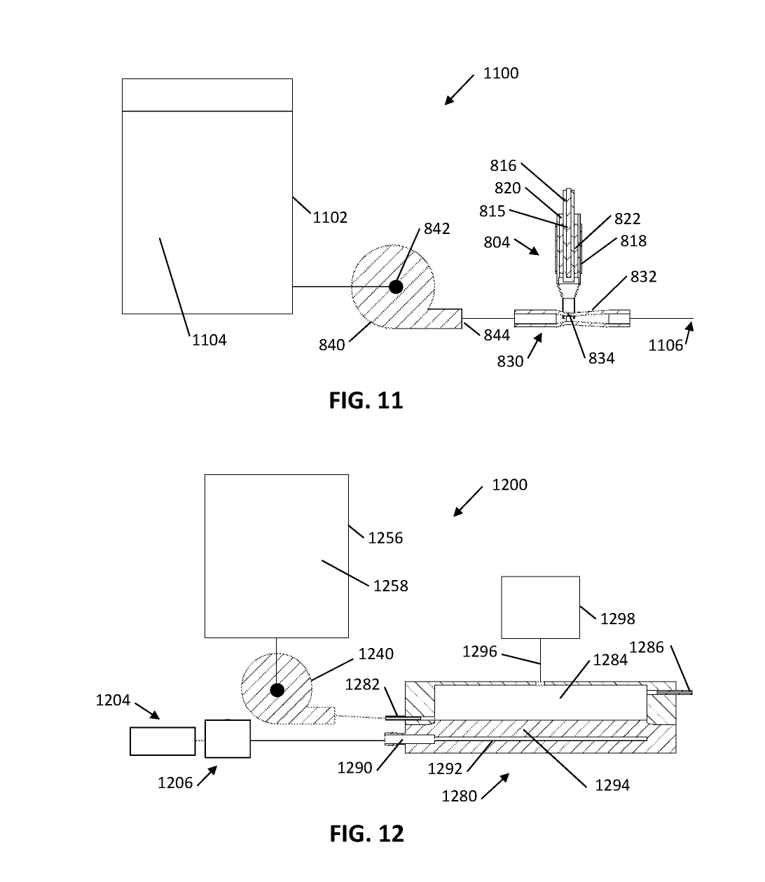

FIG. 11 is another exemplary embodiment of a large volume plasma activated liquid generating system 1100 using a venturi device 830. System 1100 includes a tank 1102 of liquid 1104, pump 840, plasma generator 804 and venturi tube 830. This embodiment is similar to those described above, except the plasma activated fluid flowing out of outlet 1106 is not reticulated back into the tank, but rather discharged through the outlet 1106 for use in decontaminating a surface.

FIG. 12 is an exemplary embodiment of a large volume plasma activated liquid generating system 1200 using a bubbler device 1280. System 1200 includes a tank 1256, liquid pump 1240, an air pump 1204, a plasma generator 1206, a gas bubbler 1280 and an ozone destruction device 1298. Tank 1256 holds a volume of liquid 1258 to be activated. The liquid may be any type of liquid, such as, for example, those described above. The liquid is pumped out of tank 1256 by pump 1240. Pump 1240 pumps liquid 1258 into reservoir 1284 in bubbler 1280 through liquid inlet 1282. An air pump 1204 pumps gas through a plasma generator 1206. Plasma generator 1206 may be any type of plasma generator, such as, for example, those described above or incorporated herein. The gas is activated by plasma generator 1206 and is pumped into inlet 1290 of bubbler 1280. Bubbler 1280 includes a diffuser 1294. The plasma activated gas flows up from passage 1292 through diffuser 1294 in the form of micro-bubbles. The micro-bubbles of activated gas flows into the liquid to be activated. Excess gas in reservoir 1284 flows up through conduit 1296 into ozone destruction unit 1298 and exhausts into the atmosphere. In this exemplary embodiment, the gas is air, however, the gas may be any gas, such as, for example, those described herein.

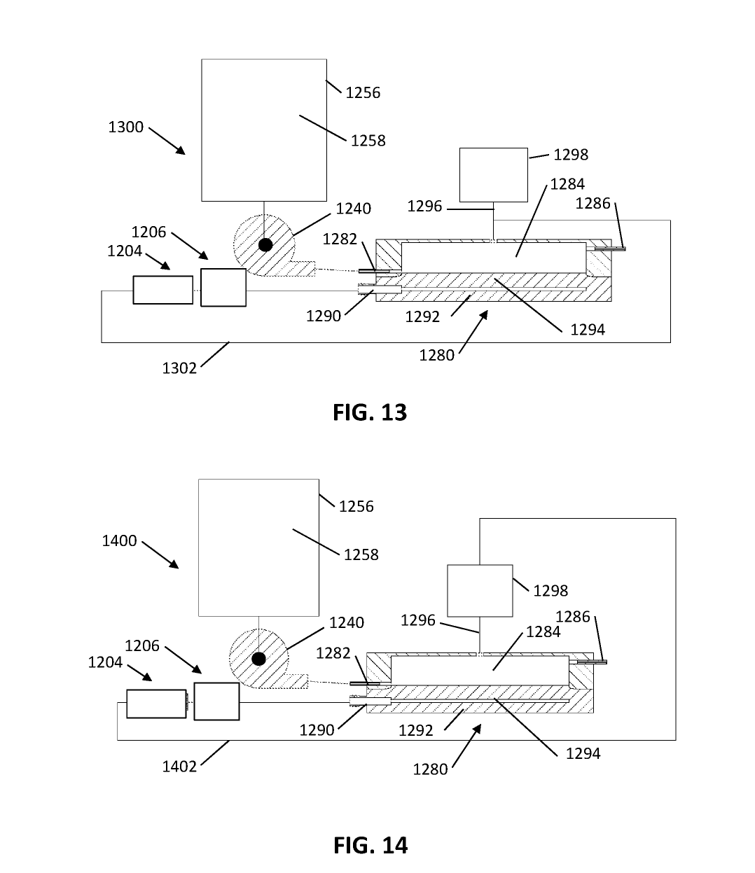

FIG. 13 is another exemplary embodiment of a large volume plasma activated liquid generating system 1300 using a bubbler device 1280. System 1300 is similar to system 1200 and like components are identified with the same numerals and are not re-described herein. System 1300 includes a conduit 1302 that connects conduit 1284 to the inlet of air pump 1204 to recirculate at least a portion of the gas. In some embodiments, a valve (not shown) is provided to control the volume of gas recirculated through the system. An air inlet (not shown), and any necessary valving, may also be added so that the mixture of recirculated gas and air, or other selected gas, can be controlled. In some embodiments disclosed herein, using recirculated gas allows for higher concentrations of active species due to the gas already having some reactive species.

FIG. 14 is another exemplary embodiment of a large volume plasma activated liquid generating system 1400 using a bubbler device 1280. System 1400 is similar to system 1400 and like components are identified with the same numerals and are not re-described herein. System 1400 includes a conduit 1402 that connects the outlet of ozone destruction unit to the inlet of air pump 1204. In some embodiments, a valve (not shown) is provided to control the volume of gas recirculated through the system. An air inlet (not shown), and any necessary valving, may also be added so that the mixture of recirculated gas and air, or other selected gas, can be controlled. In this exemplary embodiment, the amount of ozone does not build up as it may in system 1300. In some embodiments, using recirculated gas allows for higher concentrations of active species due to the gas already having some reactive species.

Generating high concentrations of activated species in a plasma activated liquid is very desirable especially when trying to kill microbes, spores, etc. that are difficult to kill, such as C-diff. Of course, this plasma activated liquid could also be used to kill many other undesirable organisms as well. Typically, the higher the concentration of active species in plasma activated liquid, the shorter the kill time is. Moreover, high volumes of the activated liquid are required in short periods of time when commercially decontaminating surfaces.

While the present invention has been illustrated by the description of embodiments thereof and while the embodiments have been described in considerable detail, it is not the intention of the applicants to restrict or in any way limit the scope of the appended claims to such detail. For example, while the embodiments illustrate methods and system that activate liquid by mixing liquid with activated gas or condensing highly activated liquid vapor or droplets, any method that can be used to condense highly activated vapor or droplets (such as high pressure or cold temperatures) and any method that can be used to inject gas into liquid (such as high pressure nozzles under the surface of the liquid injecting plasma activated gas into a liquid) may be used. Additional advantages and modifications will readily appear to those skilled in the art. Moreover, elements described with one embodiment may be readily adapted for use with other embodiments. Therefore, the invention, in its broader aspects, is not limited to the specific details, the representative apparatus and/or illustrative examples shown and described. Accordingly, departures may be made from such details without departing from the spirit or scope of the applicants' general inventive concept.

We claim:

1. A system for creating a large volume of plasma-activated liquid, comprising:
a gas circulator that moves a gas through the system;
a plasma generator for generating plasma to activate the gas to form an activated gas;
a supply of liquid to be activated;
a liquid aerator for creating an aerated liquid;
an activation chamber for activating the aerated liquid by contacting the aerated liquid with activated gas forming an activated liquid gas mixture; and
a cyclonic separator positioned downstream of the activation chamber; wherein the cyclonic separator separates at least a portion of the activated liquid gas mixture into an activated liquid and the gas;
wherein the activated liquid is collected in a container.

2. The system of claim 1 wherein the aerated liquid is one of a mist, a fog and droplets of liquid.

3. The system of claim 1 wherein the liquid aerator is a piezoelectric element.

4. The system of claim 1 wherein the gas is recirculated through the system.

5. The system of claim 4 wherein the recirculated gas contains liquid particles entrained in the gas.

6. The system of claim 1 wherein the plasma generator is a dielectric barrier discharge plasma generator.

7. The system of claim 1 wherein the plasma generator is a corona discharge plasma generator.

8. The system of claim 1 wherein the gas is air.

9. The system of claim 1 further comprising a second plasma generator, wherein the second plasma generator is located between the activation chamber and the cyclonic separator and wherein the second plasma generator generates plasma that contacts the liquid gas mixture.

10. A system for creating a large volume of plasma-activated liquid comprising:
a gas circulator that moves a gas;
a plasma generator for generating plasma to activate the gas to form an activated gas;
a supply of liquid to be activated;
a liquid aerator for creating an aerated liquid;
an activation chamber for activating the aerated liquid by contacting the aerated liquid with activated gas forming an activated liquid gas mixture; and
a liquid gas separator positioned downstream of the activation chamber; wherein the liquid gas separator separates at least a portion of the activated liquid gas mixture into an activated liquid and gas; and
wherein the activated liquid flows out of a first portion of the liquid gas separator and the gas flows out of a second portion of the liquid gas separator.

11. The system of claim 10 wherein the liquid gas separator utilizes centrifugal force to separate the liquid from the gas.

12. The system of claim 11 wherein the liquid gas separator utilizes centrifugal force and gravity to separate the liquid from the gas.

13. The system of claim 10 wherein the gas is recirculated through the system.

14. The system of claim 10 wherein the gas is air.

15. The system of claim 13 wherein the recirculated gas contains liquid particles.

16. A system for creating a large volume of plasma-activated liquid, comprising:
a gas pump that moves a gas and liquid entrained in the gas;
one or more plasma generators for generating plasma to activate at least one of the gas and liquid entrained in the gas;
a supply of liquid to be activated;
a liquid aerator for creating an aerated liquid to be entrained in the gas;
an activation chamber for activating the aerated liquid by contacting at least one of the aerated liquid or aerated liquid entrained in gas with plasma or plasma activated gas forming an activated liquid gas mixture; and
a liquid gas separator positioned downstream of the activation chamber; wherein the liquid gas separator separates at least a portion of the activated liquid gas mixture into an activated liquid and the gas; and
wherein the activated liquid flows out of a first portion of the liquid gas separator and the gas flows out of a second portion of the liquid gas separator.

17. The system of claim 16 wherein the liquid gas separator is a cyclonic separator.

18. The system of claim 16 wherein the liquid aerator is one or more piezoelectric discs.

19. The system of claim 16 wherein at least a portion of the gas is recirculated.

20. The system of claim 19 wherein the at least a portion of the gas contains liquid.
